(19) United States
(12) Patent Application Publication
(10) Pub. No.: US 2003/0152931 A1
(43) Pub. Date: Aug. 14, 2003

(54) Nucleic acid detection device and method utilizing the same

(76) Inventors: Chung-Fan Chiou, Hsinchu (TW); Chuan-Hsiung Chang, Hsinchu (TW); Cheng-Tao Wu, Shindian City (TW); Yao-Sung Chang, Hsinchu (TW); Jie-Len Huang, Taipei (TW); Chao-Chi Pan, Hsinchu (TW)

Correspondence Address: VENABLE, BAETJER, HOWARD AND CIVILETTI, LLP, P.O. BOX 34385, WASHINGTON, DC, 20043-9998, US

(21) Appl. No.: 10/071,087

(22) Filed: Feb. 11, 2002

(51) Int. Cl. C12Q 1/68; C12M 1/34

(52) U.S. Cl. 435/6; 435/287.2

(57) ABSTRACT

The present invention relates to a nucleic acid detection device. The present invention especially relates to a nucleic acid detection device utilizing the novel concept of multiple probes to one target. The device comprises a solid support, on which at least one probe set is attached. The probe set comprises multiple types of oligonucleotide probes, wherein each probe has a unique sequence complementary to the different target nucleic acid. The present invention also relates to a method for detecting nucleic acid. The method involves carrying out a hybridization reaction of the previously described device and a sample in order to determine the nucleic acids present in the sample.

### BACKGROUND OF THE INVENTION

1. Field of the Invention

The present invention relates to the field of nucleic acid detection. In particular, the present invention provides a nucleic acid detection system based on the hybridization of the probe and the target. The present invention also provides a method of detecting nucleic acid using the system.

2. Description of the Related Art

Two strains of nucleic acid having complementary sequences that can form a steady double stranded structure. Based on this property, many nucleic acid detection techniques have been developed. In general, the probe used in this method is either polynucleotide or oligonucleotide. These techniques, however, all employ the concept of one probe to one target detection. For example, please refer to FIG. 1, which shows a traditional microarray. The microarray comprises a solid support 10 with one surface physically divided into many addressable areas represented here as spots 11. Probes are attached to spots 11 so that each spot contains a single type of probes with the same sequence, which is complementary to the target nucleic acid.

FIG. 2 is a schematic diagram of a traditional nucleic acid detection device employing a polynucleotide as the probe. The polynucleotide probe 201 is immobilized on a solid support 200, wherein the sequence of polynucleotide probe 201 is complementary to a sequence 203 of the target nucleic acid 202. A typical polynucleotide probe such as a cDNA or a cDNA fragment can be hundreds to thousands of bases long. Such polynucleotide probes hybridize with target nucleic acids tightly even during high stringency washing procedures, and therefore achieve high detecting sensitivity. The drawback of using a polynucleotide probe is low specificity since a polynucleotide probe can hybridize with a nucleic acid even when homology is as low as 70%. Thus the result of detection using a polynucleotide probe is less reliable.

FIG. 3 is a schematic diagram of a traditional nucleic acid detection device employing an oligonucleotide as the probe. The oligonucleotide probe 301 is immobilized on a solid support 300, wherein the sequence of oligonucleotide probe 301 is complementary to a sequence 303 of the target nucleic acid 302. Oligonucleotide probes are usually 15-60 bases long. Normally, they are selected to have sequences complementary to the most specific fragments within the target nucleic acid. Oligonucleotide probes have very high specificity. When appropriate wash is applied, a single base difference can be distinguished with an oligonucleotide probe. However, the oligonucleotide probe's bond with the target nucleic acid is weak, especially when the target nucleic acid is much longer than the probe. Therefore, the captured target nucleic acid can easily detach from the oligonucleotide probe during wash procedures. Moreover, determining a long nucleic acid with a short oligonucleotide probe creates potentially insufficient reliability.

### SUMMARY OF THE INVENTION

An object of the present invention is to provide a nucleic acid detection device for detecting and identifying nucleic acids in a sample. The device is designed so that a probe set containing a plurality of oligonucleotide probes is used to detect a target nucleic acid.

Another object of the present invention is to provide a method for detecting and identifying nucleic acid with both high specificity and sensitivity.

The present invention involves mixing certain types of oligonucleotide probes in a predetermined ratio, and immobilizing the oligonucleotide probes on a solid support to form a nucleic acid detection device. The oligonucleotide probes are used for detecting and/or identifying target nucleic acid molecules in a sample. This nucleic acid detection device is contacted with sample nucleic acids to undergo hybridization. That is, allowing those sample nucleic acids with sequence fragments complementary to the probes to base pair with the probes.

The probe sets are immobilized in a unique addressable area on the solid support. When the hybridization between sample nucleic acids and the probes is completed, the result is determined according to detection signals attached on the sample nucleic acid and the corresponding position of each probe set. Based on the result, the existence and quantity of certain nucleic acids in the sample is estimated.

The nucleic acid detection device and method of the present invention is a very powerful tool in research as well as diagnosis applications in, for example, gene expression profiling, target identification, SNP screening, genotyping, sequencing, fingerprinting, mapping biological macromolecules, and disease diagnosis. Other applications include environmental, safety, and quality control.

Definitions

As used herein, “probe” refers to a determinable nucleic acid molecule capable of binding to a fragment of a target nucleic acid of complementary sequence through one or more types of chemical bonds, usually through hydrogen bond formation. A probe may be composed of nucleic acids capable of paring with DNA or RNA, such as DNA, RNA, and PNA.

A “probe set” is a group of different types of oligonucleotides. Each type of oligonucleotide has a determinable sequence that is different from the sequences of the other types of oligonucleotides in the same probe set.

An “oligonucleotide” herein describes an unbranched polymer of nucleotide, which is any of various compounds consisting of a nucleoside combined with a phosphate group and forming the basic constituent of DNA and RNA. Examples include, but are not limited to, DNA, RNA, and PNA.

A “target nucleic acid” typically has its origin in a defined region of the genome (for example a clone or several contiguous clones from a genomic library), or corresponds to a functional genetic unit, which may or may not be complete (for example a full or partial cDNA). The target nucleic acid can also comprise inter-Alu or Degenerate Oligonucleotide Primer PCR products derived from such clones.

As for the purpose of the present invention, a target nucleic acid may include a variety of different DNA sequences sharing a common specific region or homologue of interest. Therefore, in a probe set of the present invention, probes are complementary to different target sequences of the target nucleic acid, wherein the target nucleic acid could be more than one nucleic acid of, for example, different members of a gene family or homologues from different species.

The target nucleic acid may, for example, contain specific genes or be from a chromosomal region suspected of being present at increased or decreased copy numbers in cells of interest, e.g., tumor cells. The target element may also contain mRNA, or cDNA derived from such mRNA, suspected of being transcribed at abnormal levels.

Alternatively, a target nucleic acid may be of unknown significance or location. A sample can represent locations, either continuously or at discrete points, any desired portion of a genome, including, but not limited to, an entire genome, a single chromosome, or a portion of a chromosome.

### BRIEF DESCRIPTION OF THE DRAWINGS

FIG. 1 is a schematic diagram of a traditional microarray.

FIG. 2 is a schematic diagram of a traditional nucleic acid detection device employing a polynucleotide as the probe.

FIG. 3 is a schematic diagram of a traditional nucleic acid detection device employing an oligonucleotide as the probe.

FIG. 4 is a schematic diagram of a nucleic acid detection device of the present invention employing a plurality of oligonucleotides as the probe, wherein each oligonucleotide probe is complementary to a target sequence of the target nucleic acid.

FIG. 5 is a schematic diagram of a microarray of the present invention.

FIG. 6 is a schematic diagram of microparticles of the present invention.

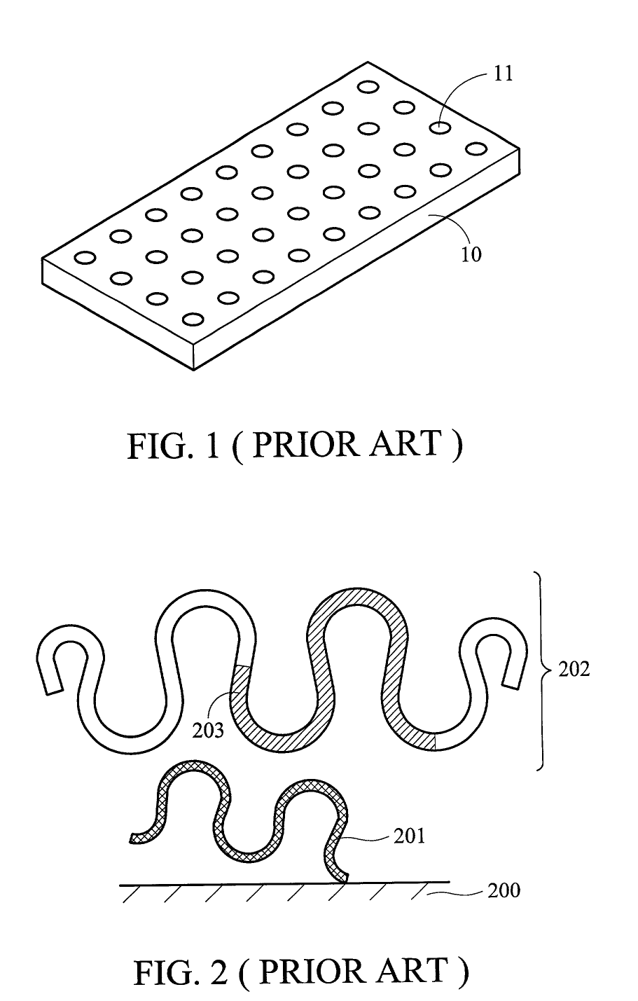

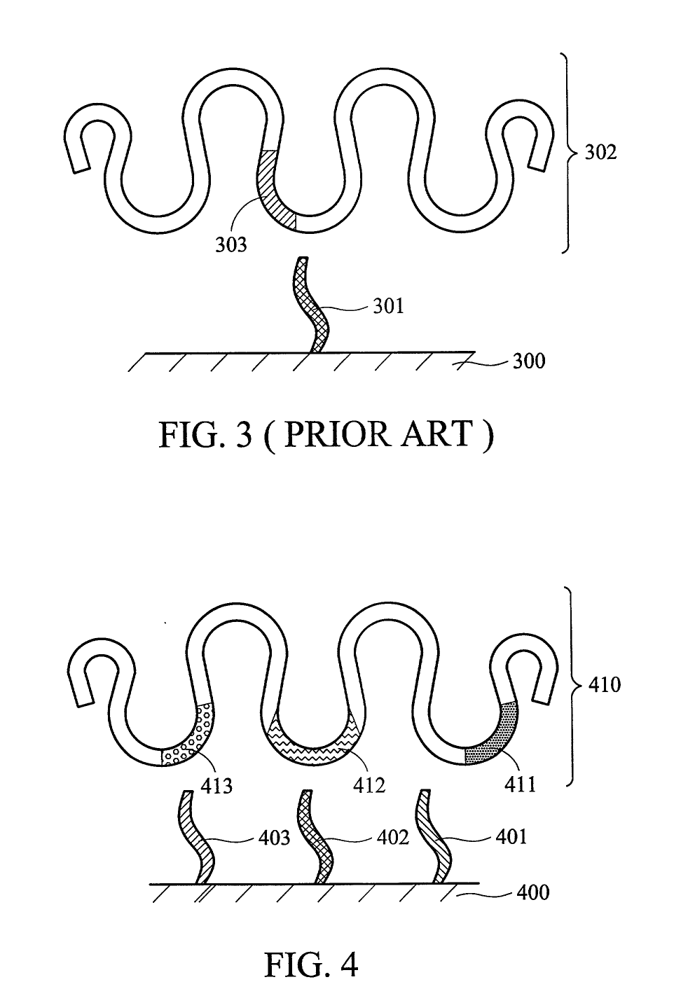

### DETAILED DESCRIPTION OF THE PREFERRED EMBODIMENT

The main feature of the present invention is its use of more than one probe to capture a common target nucleic acid. FIG. 4 is a schematic diagram of this novel concept. On a solid support 400, probes 401, 402, and 403 are immobilized. The target nucleic acid 410 contains sequences 411, 412, and 413. Each of probes 401, 402, and 403 has a unique and determinable sequence complementary to target sequences 411, 412, and 413 respectively. Target nucleic acid 410 is captured when successfully hybridizing with any one of probes 401, 402, or 403. Target nucleic acid 410 can also hybridize with any two or three of the probes. The present invention avoids low specificity by using more specific oligonucleotides as the probes rather than a long sequence polynucleotide. At the same time, detection sensitivity is increased due to the increased probability for the target to hybridize with the probes. The nucleic acid detection device of the present invention comprises a solid support, on which one or a plurality of probe sets is immobilized. The solid support can be, for example, a glass slide, a tube, a fiber, a microparticles or microbeads. If the surface of the solid support is porous, the probe sets can be immobilized inside the apertures. A gel material can also be applied to the solid support if desired, and the probe sets are immobilized on the gel material or inside it. The probe sets are immobilized on/in a unique addressable area on at least one surface of the solid support. The surface is physically divided into a plurality of unique addressable areas such that the probe sets are separately immobilized to different addressable areas. The size of the addressable area is variable according to the purpose. The size of the addressable area can be, for example, smaller than 1 mm²₁ preferably smaller than 0.1 mm², more preferably smaller than 0.01 mm², and even more preferably smaller than 0.0001 mm². The nucleic acid detection device of the present invention can also comprise one or more single nucleotide probes along with the probe set(s) in different addressable areas if desired. A probe set contains multiple types of oligonucleotides used as probes. Each oligonucleotide probe has a determinable sequence different from the other oligonucleotide probe(s) Within a probe set, the oligonucleotide probes have sequences complementary to different target nucleic acid. For example, different types of oligonucleotide probe within a probe set recognize common target through different fragments of the target. Or different types of oligonucleotide probe within a probe set recognize different conserved sequences. The ratio of different oligonucleotide probes present in a probe set is adjusted according to the conditions under which the procedure is carried out. For example, a substantially equal quantity of each probe is preferred in some cases, and in other cases, adjusting ratio of the probes according to their affinity to the target is desired.

The oligonucleotide probes are nucleic acids capable to pair up with DNA or RNA, for example, DNA, RNA, and PNA (peptide nucleic acid).

The method for detecting nucleic acid of the present invention comprises the following steps. First, a plurality of probe sets comprising multiple oligonucleotide probes as previously described is provided, as is a solid support having at least a unique addressable area. Each of the probe sets is attached to a unique addressable area of the solid support. Probes are contacted with samples containing nucleic acids for enough time to proceeding hybridization reaction. The sample nucleic acids are labeled with a detectable signal before or after the hybridization, and the result of the hybridization is determined through this labeling system.

The labeling of the sample nucleic acids can be achieved by any conventional labeling technique used in biological molecule detection, such as fluorescence labeling, chemiluminescence labeling, electric signal labeling, or radioactive labeling.

According to Keller and Manak (G. H. Keller and M. M. Manak; DNA Probes; second edition; MacMillan Pub. Ltd.; pp. 1-25, 1993), factors affecting the stability of a hybrid of two nucleic acids include temperature, length of sequences, GC contents of the sequences, degree of complement between the two nucleic acids, and reacting environments such as salt.

The efficiency of using single probe and using three probes to detect a target nucleic acid in a sample containing nucleic acids with more than one sequence is compared in this example. All involved probes have been designed to be complementary to the target but not complementary to any background nucleic acid. In FIG. 4, three specific sequences (411, 412, and 413) are selected from the target, and their complementary sequences are used as the probes, 401, 402, and 403. Assuming suitable conditions, a probe has a 60% chance to correctly hybridize with the target. When detection is carried out in the traditional one probe to one target fashion, ie., using any of probes 401, 402, and 403 to hybridize with the target, 60% of the target will remain attached to the probe after wash. When all three probes are used, target nucleic acids hybridizing with any one of, any two of, or all three of the probes are immobilized on the solid support and thus isolated from the other nucleic acids. The probability of different situations of hybridization between the target nucleic acids and the probes is as follows:

Target nucleic acid hybridize with three of the probes:

P_{b1}=(0.6×0.6×0.6)=0.216

Target nucleic acid hybridize with two of the probes:

P_{b2}=(0.6×0.6×0.4)×3=0.432

Target nucleic acid hybridize with one of the probes:

P_{b3}=(0.6×0.4×0.4)×3=0.288

Target nucleic acid hybridize with none of the probes:

P_{b0}=(0.4×0.4×0.4)=0.064

Therefore, detecting a target with three probes captures as much as 93.6% of the target nucleic acids present in the sample compared with 60% of single-probe detection.

The present invention not only increases the ability of target capture, but also avoids non-specific hybridization that often occurs when the whole complementary sequence is used as a probe. The present invention thus provides a novel concept for nucleic acid detection with both high sensitivity and high specificity.

The present invention therefore provides a very powerful tool for research, diagnosis, and other applications involving nucleic acid detection, for example, in gene expression profiling, target identification, SNP screening, genotyping, sequencing, fingerprinting, mapping biological macromolecules, or disease diagnosis, as well as environmental, safety, and quality control.

Preferred Embodiment 1:

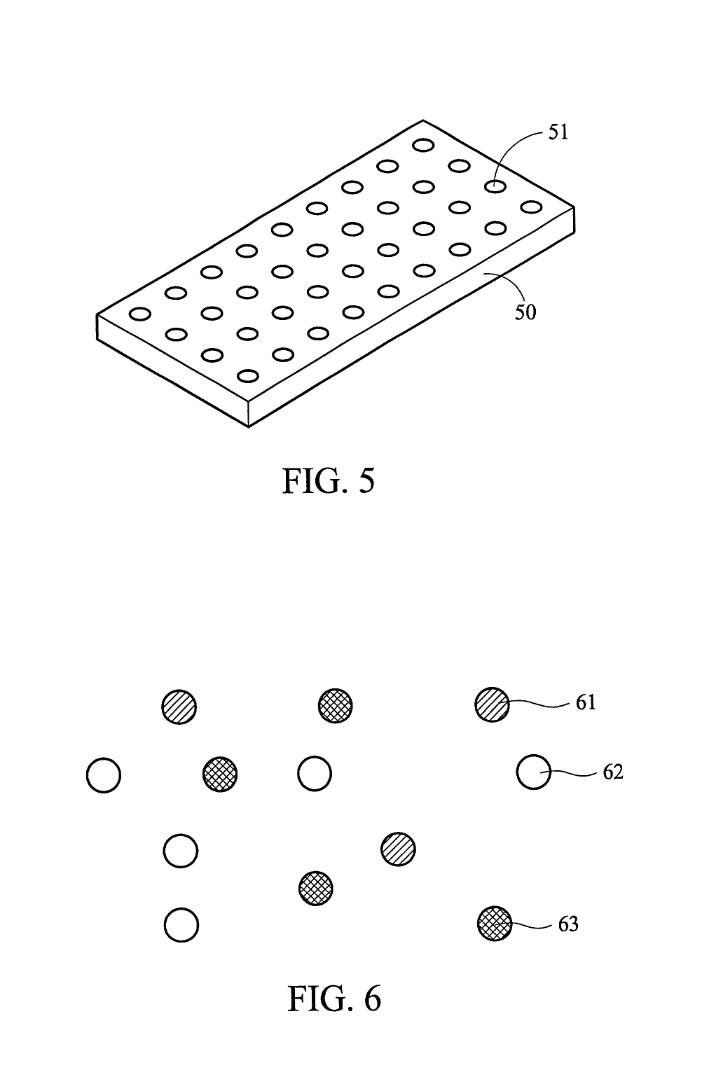

Please refer to FIG. 5. Preferred embodiment of the present invention is a microarray having the previously described probe sets. The solid support 50 of the microarray is a chip such as a glass chip, a silicon chip, or a film of suitable material. The detection surface of the chip is physically divided into multiple unique addressable areas 51 each preferably smaller than 1 mm², more preferably smaller than 0.1 mm², further more preferably smaller than 0.01 mm², even more preferably smaller than 0.0001 mm².

On each of unique addressable areas 51, a probe set is immobilized. Each probe set comprises more than one type of oligonucleotide probe having different sequences, preferably 2 to 10 types, more preferably 2 to 4 types. Each type of the oligonucleotide probes has a determinable sequence, and is preferably specific to a different fragment of the target. The length of each oligonucleotide probe is preferably 4 to 400 bases, more preferably 8 to 80 bases.

The probe sets are immobilized on the surface of solid support 50, or inside if solid support 50 is porous. A gel material can also be applied to solid support 50, and the probe sets is immobilized on the gel material or within it. The density of the probe sets in addressable area 51 on solid support 50 is preferably greater than 100 probe sets (or addressable areas) per cm², more preferably greater than 1,000 probe sets per cm² further preferably greater than 10,000 probe sets per cm². The oligonucleotide probes are nucleic acids capable of pairing with DNA or RNA, for example DNAs, RNAs, or PNAs. Preferably, each probe set contains substantially equal quantities of each type of oligonucleotide, or when desired, quantities of each type of oligonucleotides correspond to affinity to the target sequence. Moreover, probes within a probe set are preferably evenly distributed across their designated unique addressable area.

Preferred Embodiment 2:

Another preferred embodiment of the present invention is a nucleic acid detection device comprising microbeads attached to the previously described probe sets. FIG. 6 illustrates a schematic diagram of a mixture of microparticles described herein. Three types of microparticles each have their own identification and are shown as microparticles 61, 62, and 63. Each type of the microparticles is attached to assigned probe set. Different microparticles can be used separately, or more preferably, used as a mixture as shown in the figure to detect different targets in a sample.

The particle is glass, polymer, plastic, metals, silicon, or inorganic materials. The microparticles carry identification of their own, for example, the identification can be particle size, fluorescence signal, radioactive signal, shape of the particle, color of the particle, or composition. A probe set is immobilized to one or a collection of the microbeads with the same identification. Therefore, when microparticles with different identification with different probe sets respectively are used in a mixture, the reaction results of different probe sets are observed according to the identification of the microparticles. The probe sets can be immobilized on the surface of the microparticles, or inside the microparticles when the microparticles are porous. If desired, a gel material can be applied to the microparticles, and the probe sets are immobilized on the gel material or within it.

Each of the previously mentioned probe sets comprises more than one type of oligonucleotide probes having different sequences, preferably 2 to 10 types, more preferably 2 to 4 types. Each type of oligonucleotide probe has a determinable sequence, and is preferably specific to a different fragment of the target. The length of each oligonucleotide probe is preferably 4 to 400 bases, more preferably 8 to 80 bases. The oligonucleotide probes are nucleic acids capable of pairing with DNA or RNA, for example DNAs, RNAs, or PNAs. Preferably, each probe set contains substantially equal quantities of each type of oligonucleotide, or when desired, quantities of each type of oligonucleotides correspond to affinity to the target sequence. Moreover, probes within a probe set are preferably evenly distributed across their designated unique addressable area.

Preferred Embodiment 3:

Another preferred embodiment of the present invention is a method for detecting nucleic acid involving the use of the previously described nucleic acid detection device. The feature of the method is the application of probe sets each with different types of oligonucleotide probes as previously described. The method comprises the steps of providing a plurality of the probe sets and the solid support, also previously discussed. The probe sets are attached to the solid support by conventional methods. Sample nucleic acids are than subjected to hybridize with the probe sets. The sample nucleic acids are labeled with a detectable signal before or after the hybridization. The hybridization results are determined through the signal on the sample nucleic acids with conventional methods.

The labeling of the sample nucleic acids is achieved by performing fluorescence labeling, chemiluminescence labeling, electric signal labeling, or radioactive labeling.

The oligonucleotide probes can either be synthesized and then immobilized on the solid support, or be directly synthesized on the solid support. Different types of oligonucleotide probes within a probe set are immobilized in sequence or immobilized simultaneously as a mixture, and are evenly distributed across the unique addressable area.

Finally, while the invention has been described by way of example and in terms of the preferred embodiment, it is to be understood that the invention is not limited to the disclosed embodiments. On the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A nucleic acid detection device, comprising:
a solid support having at least one unique addressable area;
at least one probe set, each comprising multiple types of oligonucleotides, each type of oligonucleotide having a determinable sequence, wherein the oligonucleotides are immobilized on at least one surface of the solid support, each probe set being localized in one of the addressable areas.

2. The device as set forth in claim 1, wherein each probe set comprises 2 to 10 types of oligonucleotides having different sequences.

3. The device as set forth in claim 2, wherein each probe set comprises 2 to 4 types of oligonucleotides having different sequences.

4. The device as set forth in claim 1, wherein each oligonucleotide in each probe set is specific to a different target sequence.

5. The device as set forth in claim 1, wherein t he length of each oligonucleotide is 4 to 400 bases.

6. The device as set forth in claim 5, wherein the length of each oligonucleotide is 8 to 80 bases.

7. The device as set forth in claim 1, wherein each addressable area is smaller than 1 mm².

8. The device as set forth in claim 1, wherein each addressable area is smaller than 0.1 mm².

9. The device as set forth in claim 1, wherein each addressable area is smaller than 0.01 mm².

10. The device as set forth in claim 1, wherein each addressable area is smaller than 0.0001 mm².

11. The device as set forth in claim 1, wherein the density of the probe set on the solid support is greater than 100 probe sets per cm².

12. The device as set forth in claim 1, wherein the density of the probe set on the solid support is greater than 1,000 probe sets per cm².

13. The device as set forth in claim 1, wherein the density of the probe set on the solid support is greater than 10,000 probe sets per cm².

14. The device as set forth in claim 1, wherein the solid support is a plane surface, a curved surface, a tube, a fiber, a microparticle, or a microbead.

15. The device as set forth in claim 1, wherein the solid support is glass, polymer, plastic, metal, silicon, or inorganic materials.

16. The device as set forth in claim 1, wherein the probe set is immobilized on the surface of a gel material applied to the solid support or inside a gel material applied to the solid support.

17. The device as set forth in claim 1, wherein the solid support has a porous structure.

18. The device as set forth in claim 1, wherein the device is a microarray.

19. The device as set forth in claim 1, wherein the oligonucleotides are nucleic acids capable of pairing with DNA.

20. The device as set forth in claim 1, wherein the oligonucleotides are nucleic acids capable of pairing with RNA.

21. The device as set forth in claim 1, wherein the oligonucleotides are DNAs.

22. The device as set forth in claim 1, wherein the oligonucleotides are RNAs.

23. The device as set forth in claim 1, wherein the oligonucleotides are PNAs.

24. The device as set forth in claim 1, wherein each type of the oligonucleotide is synthesized and then immobilized on the solid support.

25. The device as set forth in claim 24, wherein each type of oligonucleotide is immobilized in sequence.

26. The device as set forth in claim 24, wherein each type of oligonucleotide is immobilized simultaneously as a mixture.

27. The device as set forth in claim 1, wherein each type of oligonucleotide is directly synthesized on the solid support.

28. The device as set forth in claim 1, wherein each probe set contains substantially equal quantities of each type of the oligonucleotide.

29. The device as set forth in claim 1, wherein the oligonucleotides of a probe set are evenly distributed across the unique addressable area.

30. The device as set forth in claim 1, wherein the quantity of each type of oligonucleotides corresponds to its affinity to the target sequence.

31. A nucleic acid detection device, comprising:
at least one type of distinguishable microparticle, each distinguishable from other types of microparticles;
at least one probe set, each comprising multiple types of oligonucleotides designed specifically to different target sequences, each type of oligonucleotide having a determinable sequence, wherein the oligonucleotides are immobilized on the microparticles.

32. The device as set forth in claim 31, wherein each type of microparticle carries identification distinguishing it from other types of microparticles, the identification based on particle size, fluorescence signal, radioactive signal, shape, color, or composition of the particle.

33. The device as set forth in claim 31, wherein the particle is glass, polymer, plastic, metal, silicon, or inorganic materials.

34. The device as set forth in claim 31, wherein the probe sets are immobilized on the surface of the microparticles.

35. The device as set forth in claim 31 wherein the microparticles are porous, and the probe sets are immobilized inside the microparticles.

36. The device as set forth in claim 31, wherein the probe sets are immobilized on the surface or inside a gel material applied to the microparticles.

37. The device as set forth in claim 31, wherein each probe set comprises 2 to 10 types of oligonucleotides having different sequences.

38. The device as set forth in claim 37, wherein each probe set comprises 2 to 4 types of oligonucleotides having different sequences.

39. The device as set forth in claim 31, wherein the length of each oligonucleotide is 4 to 400 bases.

40. The device as set forth in claim 39, wherein the length of each oligonucleotide is 8 to 80 bases.

41. A method for detecting nucleic acid, comprising the following steps:
providing at least one probe set comprising multiple types of oligonucleotides;
providing a solid support having at least one unique addressable area;
attaching the probe set to the unique addressable area of the solid support;
allowing sample nucleic acids to hybridize with probe sets;
labeling the sample nucleic acids with a detectable signal before or after the hybridization; and
determining the results of the hybridization.

42. The method as set forth in claim 41, wherein the labeling of the sample nucleic acids is achieved by performing a technique fluorescence labeling, chemiluminescence labeling, electric signal labeling, and radioactive labeling.

43. The method as set forth in claim 41, wherein each probe set comprises 2 to 10 types of oligonucleotides having different sequences, and each type of oligonucleotide specific to a different target sequence.

44. The method as set forth in claim 43, wherein each probe set comprises 2 to 4 types of oligonucleotides having different sequences, and each type of oligonucleotide specific to a different target sequence.

45. The method as set forth in claim 41, wherein the length of each oligonucleotide is 4 to 400 bases.

46. The method as set forth in claim 45, wherein the length of each oligonucleotide is 8 to 80 bases.

47. The method as set forth in claim 41, wherein the oligonucleotides are nucleic acids capable of pairing with DNA.

48. The method as set forth in claim 41, wherein the oligonucleotides are nucleic acids capable of pairing with RNA.

49. The method as set forth in claim 41, wherein the oligonucleotides are DNAs.

50. The method as set forth in claim 41, wherein the oligonucleotides are RNAs.

51. The method as set forth in claim 41, wherein the oligonucleotides are PNAs.

52. The method as set forth in claim 41, wherein each probe set contains substantially equal quantities of each type of the oligonucleotides.

53. The method as set forth in claim 41, wherein the oligonucleotides of a probe set are evenly distributed across the unique addressable area.

54. The method as set forth in claim 41, wherein the quantity of each type of the oligonucleotides corresponds to its affinity to the target sequence.
